(19) United States
(12) Patent Application Publication
(10) Pub. No.: US 2016/0296752 A1
(43) Pub. Date: Oct. 13, 2016

(54) PULSE POLARITY SWITCHING FOR IMPROVED HUMAN BODY STIMULATION AND HEALING

(71) Applicant: NES HEALTH, LTD., Poole Dorset (GB)

(72) Inventor: Harry MASSEY, Aptos, CA (US)

(73) Assignee: NES HEALTH, LTD., Poole Dorset (GB)

(21) Appl. No.: 14/777,918

(22) PCT Filed: Mar. 14, 2014
(86) PCT No.: PCT/EP2014/000691

§ 371 Date: Sep. 17, 2015

Related U.S. Application Data
(60) Provisional application No. 61/790,806, filed on Mar. 15, 2013.

(51) Int. Cl. A61N 1/32; A61N 1/36; A61N 2/00; A61N 1/04

(52) U.S. Cl. CPC A61N 1/326; A61N 1/0468; A61N 1/36014; A61N 2/004

(57) ABSTRACT

A technique, method and associated apparatus for reversing the direction of current flow between electrodes on an electrical stimulation device placed on the skin, including controllably changing the polarity of a pulse at the electrodes. Software controlled electronics enable polarity reversing, i.e., a pulse trigger stream may be used to determine when a pulse is generated at the electrodes, and a polarity data stream may be used to determine the polarity of the pulse at the electrodes. The apparatus may include identical windings in a transformer thereof, for delivering the pulses at the electrodes. The invention may be used for bio-electro stimulation to deliver improved stimulus for human body stimulation.

### RELATED APPLICATION INFORMATION

This PCT application claims the benefit of U.S. provisional patent application Ser. No. 61/790,806, filed Mar. 15, 2013, which is hereby incorporated herein by reference in its entirety.

### TECHNICAL FIELD

The present invention relates to polarity switching, in particular, polarity switching for bio-electro stimulation and magnetic fields to deliver the stimulus for human body stimulation and healing.

### BACKGROUND OF THE INVENTION

Electrical stimulation devices generate high voltage, low current pulses that are applied to the skin using a pair of electrically conductive electrodes. This type of stimulation is relevant to bio-electro stimulation and magnetic fields to deliver the stimulus for human body stimulation and healing. Improved stimulation devices are needed, as provided by the present invention.

### SUMMARY OF THE INVENTION

The shortcomings of the prior art, and additional advantages are provided by, the present invention which in one aspect includes a technique, method and apparatus for reversing the direction of current flow between the electrodes on an electrical stimulation device placed on the skin, and may be achieved by changing the polarity of the pulse on the electrodes, instead of physically swapping the electrode positions. This is referred to as polarity switching and may be done with software controlled electronics. This type of stimulation is relevant to bio-electro stimulation and magnetic fields to deliver the stimulus for human body stimulation and healing.

All known previous devices require the user to swap the electrodes manually in order to achieve this.

Switching polarity may be controlled by changing a software data value. If the value is ‘1’ then the pulse polarity is positive; if the value is ‘0’ then the pulse polarity is negative.

A sequence of changes of polarity may be controlled by reading a sequence of data values. These data values are derived from an integer number or by toggling the data value at regular timed intervals.

In that regard, the present invention in one aspect is a technique, method and associated apparatus for reversing the direction of current flow between electrodes on an electrical stimulation device to be placed on the skin, including controllably changing the polarity of a pulse at the electrodes. Software controlled electronics enable polarity reversing, i.e., a pulse trigger stream may be used to determine when a pulse is generated at the electrodes, and a polarity data stream may be used to determine the polarity of the pulse at the electrodes. The apparatus may include identical windings in a transformer thereof, for delivering the pulses at the electrodes. This invention may be used for bio-electro stimulation to deliver improved stimulus for human body stimulation.

Additional features and advantages are realized through the techniques of the present invention. Other embodiments and aspects of the invention are described in detail herein and are considered a part of the claimed invention.

### BRIEF DESCRIPTION OF THE DRAWINGS

The subject matter regarded as the invention is particularly pointed out and distinctly claimed at the conclusion of the specification. The foregoing and other objects, features, and advantages of the invention are apparent from the following detailed description taken in combination with the accompanying drawings in which:

FIG. 1 depicts a polarity switching apparatus, in accordance with one or more aspects of the present invention; and

FIGS. 2a-b depict exemplary output waveforms, in accordance with one or more aspects of the present invention.

### DETAILED DESCRIPTION OF THE INVENTION

### Hardware

The electronics used to generate pulses described above may include a conventional transformer with a primary winding of a small number of turns and a secondary winding of a large number of turns. The primary winding may be used to charge the transformer core using a low DC voltage. Charge time may be usually less than 1 ms. On removing the primary voltage the secondary winding generates a single high voltage pulse.

The amplitude of the pulse can be controlled by varying the charge time of the primary winding.

A sequence of pulses, typically up to 1000 pulses/second, may be generated. In practice, a pulse train can vary between 15 Hz (pulses/second) and 500 Hz.

The pulse may be applied to one of the electrodes and may be either of positive or negative polarity but may be of the same polarity. The other electrode may be connected to the ground (0V) of the device.

When the electrode pair is applied to the skin the current will flow predominantly in one direction.

Reversing the direction of current flow may enhance scar tissue healing, possibly achieved by turning the electrodes around (by rotating 180 degrees).

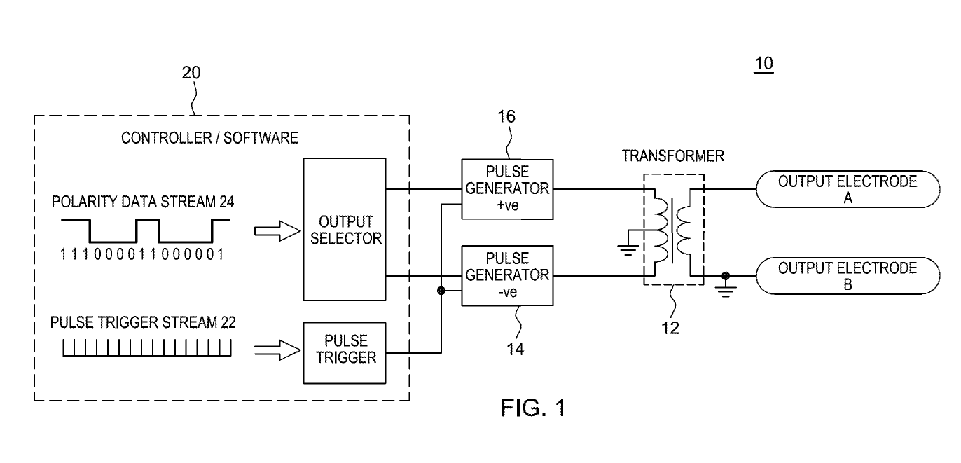

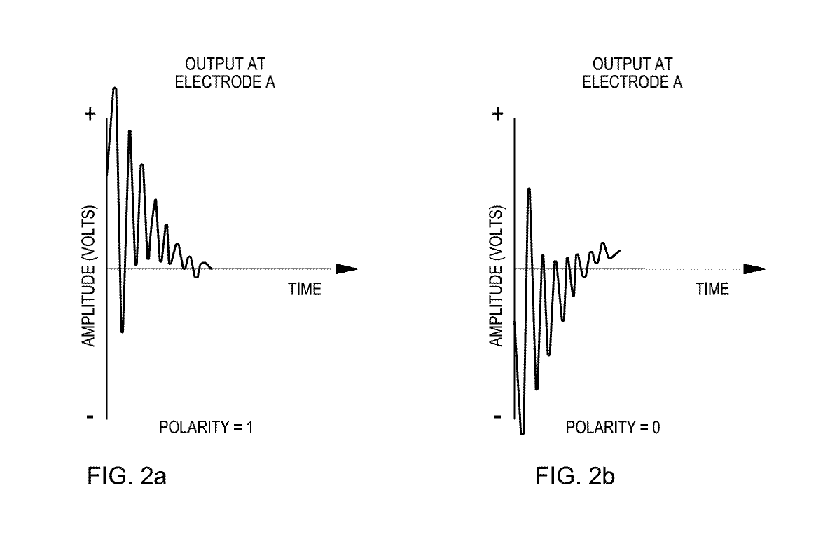

In accordance with the present invention, and with reference to the pulse generating, polarity switching apparatus 10 as shown FIG. 1, a secondary, identical primary winding may be added to a pulse generating transformer 12 thereof. This winding may be charged by applying the volts in the opposite direction in order to generate a high voltage in the secondary winding of the opposite polarity. Each primary winding may be connected to its own electronic charging circuit (including e.g., pulse generators 14, 16) under separate software control, as provided by controller/software 20 implemented in the apparatus. The apparatus may be operated as follows:
The pulse trigger stream 22 controls when output pulses on electrode A are created.
The polarity data stream 24 controls the initial voltage polarity of the output pulse.
A positive pulse is created when the polarity data is 1; a negative pulse is created when the polarity data is 0.
A positive pulse has an output voltage that is initially +ve. The resulting waveform remains predominantly +ve. (see FIG. 2a).
A negative pulse has an output voltage that is initially −ve. The resulting waveform remains predominantly −ve. (see FIG. 2b).
Both polarity and pulse trigger streams may be controlled by software to create any pattern and frequency required.

Software may then be used to select which primary winding may be operated. Only one primary winding may be operated at any one time.

### Software

With reference to FIG. 1, (POINT 1) to control the polarity 24 a software procedure (in controller/software 20) may be given a data value, which will be either ‘0’ or ‘1’. A value of ‘1’ may be interpreted to be a positive pulse and the procedure will, therefore, operate the positive primary winding in the transformer. Conversely a data value of ‘0’ may be interpreted as a negative pulse and will operate the negative primary winding.

Control of the polarity of each pulse may be done in one of two ways: (a) by toggling the polarity data value between 0 and 1 at regular time intervals. During each time interval, all pulses are output with the same polarity; and/or (b) by changing the polarity of consecutive pulses according to a sequence of data values.

Controlling polarity at a regular time interval: The operator of the device can adjust the duration of the time interval. The duration can be adjusted while the device is in use.

Controlling polarity using a sequence: A sequence of data values (0 or 1) may be presented to the controlling software. Up to 120 values in length, although there is no practical limit to the length.

(POINT 2) Each value in this sequence of 120 values may be applied to a sequence of consecutive pulses regardless of the frequency of the pulse stream. When the last value has been reached, the sequence may be repeated until the operator switches the unit off

Different sequences may be held in software as 30 digit integers.

When a sequence is selected to control polarity, each digit in the sequence may be converted to a 4 digit binary format. For example, the digit ‘9’ converts to ‘1001’. A 30- digit integer will convert to 120 digit binary sequence.

This binary sequence may be used to drive the transformer. As described in POINT 1 above ‘1’ will create a positive pulse and a ‘0’ will create a negative pulse.

An example of a numeric sequence conversion: the integer ‘12345678’ converts to: 0001 0010 0101 0100 0101 0110 0111 1000.

The binary sequence may be fed to the software from right to left. Each consecutive pulse will use the next digit in the binary sequence and repeated as described in POINT 2 above.

As mentioned above, this type of polarity switching, with software controlled electronics, may be applicable to improving bio-electro stimulation and magnetic fields to deliver stimulus for human body stimulation and healing.

As will be appreciated by one skilled in the art, control aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”. Furthermore, control aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may be any non-transitory computer readable medium that is not a computer readable storage medium and that can communicate, propagate, or transport a program for use by or in connection with an instruction execution system, apparatus or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

In one example, a computer program product includes, for instance, one or more computer readable storage media to store computer readable program code means or logic thereon to provide and facilitate one or more aspects of the present invention.

Program code embodied on a computer readable medium may be transmitted using an appropriate medium, including but not limited to wireless, wireline, optical fiber cable, RF, etc., or any suitable combination of the foregoing.

Computer program code for carrying out control and/or calibration operations for aspects of the present invention may be written in any combination of one or more programming languages, including an object oriented programming language, such as Java, Smalltalk, C++ or the like, and conventional procedural programming languages, such as the “C” programming language, assembler or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to block diagrams of methods, apparatus (systems) and computer program products according to embodiments of the invention. It will be understood that the control block of the diagram can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The block diagram in the figure illustrates the architecture, functionality, and operation of possible implementations of systems, methods and computer program products according to various embodiments of the present invention. In this regard, one or more blocks in the diagram may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that one or more blocks of the diagram can be implemented by special purpose hardware-based systems that perform the specified functions or acts, or combinations of special purpose hardware and computer instructions.

In addition to the above, one or more aspects of the present invention may be provided, offered, deployed, managed, serviced, etc. by a service provider who offers management of customer environments. For instance, the service provider can create, maintain, support, etc. computer code and/or a computer infrastructure that performs one or more aspects of the present invention for one or more customers. In return, the service provider may receive payment from the customer under a subscription and/or fee agreement, as examples. Additionally or alternatively, the service provider may receive payment from the sale of advertising content to one or more third parties.

In one aspect of the present invention, an application may be deployed for performing one or more aspects of the present invention. As one example, the deploying of an application comprises providing computer infrastructure operable to perform one or more aspects of the present invention.

As a further aspect of the present invention, a computing infrastructure may be deployed comprising integrating computer readable code into a computing system, in which the code in combination with the computing system is capable of performing one or more aspects of the present invention.

As yet a further aspect of the present invention, a process for integrating computing infrastructure comprising integrating computer readable code into a computer system may be provided. The computer system comprises a computer readable medium, in which the computer medium comprises one or more aspects of the present invention. The code in combination with the computer system is capable of performing one or more aspects of the present invention.

Although various embodiments are described above, these are only examples. For example, computing environments of other architectures can incorporate and use one or more aspects of the present invention. Additionally, the network of nodes can include additional nodes, and the nodes can be the same or different from those described herein. Also, many types of communications interfaces may be used.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to explain the principles of the invention and the practical application, and to enable others of ordinary skill in the art to understand the invention through various embodiments and the various modifications thereto which are dependent on the particular use contemplated.

1. A method of reversing the direction of current flow between the electrodes of an electrical stimulation apparatus to be placed on the skin, including controllably changing the polarity of pulses at the electrodes.

2. The method of claim 1, comprising using software controlled electronics for said reversing.

3. The method of claim 1, used in combination with bio-electro stimulation to deliver stimulus for human body stimulation.

4. The method of claim 1, wherein said reversing includes using a pulse trigger stream to determine when a pulse is generated at the electrodes, and using a polarity data stream to determine the polarity of the pulse at the electrodes.

5. The method of claim 1, wherein the apparatus includes identical windings in a transformer, for delivering the pulses at the electrodes.

6. An apparatus for reversing the direction of current flow between the electrodes on an electrical stimulation device placed on the skin, including a circuit for controllably changing the polarity of a pulse at the electrodes.

7. The apparatus of claim 6, comprising software controlled electronics for said reversing.

8. The apparatus of claim 6, in combination with an apparatus for bio-electro stimulation to deliver stimulus for human body stimulation.

9. The apparatus of claim 1, further comprising a pulse trigger stream to determine when a pulse is generated at the electrodes, and a polarity data stream to determine the polarity of the pulse at the electrodes.

10. The apparatus of claim 1, wherein the apparatus includes identical windings in a transformer thereof, for delivering the pulses at the electrodes.
